(19) United States
(12) Patent Application Publication
(10) Pub. No.: US 2026/0151050 A1
(43) Pub. Date: Jun. 4, 2026

(54) APPARATUS AND METHOD OF MEASUREMENT OF INCREMENTAL CHANGES IN PARTIAL POSTURAL CONTROL

(71) Applicants: Patricia Ann Mellodge, West Hartford, CT (US); Sandra Lee Saavedra, Lebanon, OR (US)

(72) Inventors: Patricia Ann Mellodge, West Hartford, CT (US); Sandra Lee Saavedra, Lebanon, OR (US)

(21) Appl. No.: 18/563,690

(22) PCT Filed: May 23, 2022
(86) PCT No.: PCT/IB2022/000342

§ 371 Date: Nov. 22, 2023

Related U.S. Application Data
(60) Provisional application No. 63/192,041, filed on May 23, 2021.

(51) Int. Cl. A61B 5/11; A61B 5/00

(52) U.S. Cl. CPC A61B 5/1117; A61B 5/1121; A61B 5/4082; A61B 5/4848; A61B 5/6814; A61B 5/6823; A61B 5/7264; A61B 2503/06; A61B 2562/0219

(57) ABSTRACT

Herein a method and apparatus for treating a patient with a neuromotor disorder is provided. A plurality of sensors is configured to capture kinematic data from a subject for a proscribed length of time. The kinematic data is then converted into a postural score, one or more of which may lead a clinician to create or alter a treatment plan.

### CROSS REFERENCE TO RELATED APPLICATIONS

This application claims priority to and benefit of U.S. Provisional Patent Application Ser. No. 63/1920,041 filed on May 23, 2021 which is incorporated herein by reference in its entirety.

### TECHNICAL FIELD

Embodiments of the invention generally fall into at least the categories of physical therapy assistive devices; medical devices; postural measurement devices; or aides for the diagnosis or treatment of cerebral palsy and other neuromotor disabilities.

### BACKGROUND

Trunk control develops spontaneously from the head downwards in typical infants. Failure to develop trunk control cascades into a plethora of secondary complications and limitations. Children with neurologic disorders that result in deficits in trunk postural control are unable to sit upright, which contributes to scoliosis development (i.e., curvature of the spine) with significant health risks for pneumonia, pressure sores, and surgical intervention for scoliosis and hip dysplasia. Lack of trunk control also impairs the ability of a child to play, interact with the environment, and freely use their hands.

Prognosis for improvement in trunk control is poor for non-ambulatory children. Musculoskeletal growth, trunk muscle paralysis, and the lack of effective therapeutic interventions to improve the ability to sit upright leads to rapid development of scoliosis. Current evidence indicates that 100% of children with onset of spinal cord injury (SCI) before 5 years of age, regardless of injury severity, will develop scoliosis and 67% will require surgical intervention. If a child with cerebral palsy (CP) does not gain independent sitting by 4 years of age, they are not likely to develop it in their entire lifetime. For children with CP the risk of scoliosis varies from almost no risk for ambulatory children to 50% risk of moderate to severe scoliosis by age 18 for non-ambulatory children. The current medical approach to trunk control deficits and scoliosis (i.e., management by wearing a spine or back brace for external support) does not prevent the development of scoliosis or help children recover or gain control of their trunk. Spinal curves are documented by periodic (typically annual), static radiography however, the frequency and methods for obtaining images do not provide a dynamic view of intervention effectiveness or timely feedback on changes in curve progression or changes in trunk postural control for researchers and clinicians.

For children with postural deficits, the most clinically relevant information is their response to gravitational forces. However most developmental evaluations assess development of trunk control grossly, by the ability of a child to support the entire trunk upright either by propping on the arms or with hands free. For example, the Gross Motor Function Classification System (GMFCS) divides the motor skills capabilities of a children with cerebral palsy into five subjectively assessed levels. Children classified at Level I are able to run and play freely with limited impairment whereas children classified at Level V are almost totally restricted in movement and are confined to the usage of assistive devices. Population based studies have shown that children greater than 2 years of age placed in a particular level rarely change levels. Lack of specificity for measurement of partial trunk control prevents progress within and between levels. This lack of specificity in measurement has been paralleled by a lack of specificity for intervention and poor prognosis for development of trunk control in children with neuromotor disability. Thus, there is a need for trunk posture outcome measures that are readily accessible, frequent, continuous or semi-continuous; and, that document the effects of interventions, positioning equipment, and daily activity levels on trunk alignment and control.

### BRIEF SUMMARY OF THE INVENTION

It is an object of the invention to provide a plurality of sensors affixed at known anatomical reference points on a patient with a neuromotor disorder such as cerebral palsy (“CP”). Expressly contemplated are other disorders and/or diseases that may include any neurologic or musculoskeletal pathology, non-limiting examples of which can include: spinabifidia; spinal cord injury; scoliosis; arthritis in various forms; muscular dystrophies; Down's syndrome; brain injuries or malformations; spinal muscular atrophy; acute flaccid myelitis; genetic disorders; other causes of developmental delay such as hypotonia, premature birth; and, other mobility disorders and the like are all contemplated without departing from the spirit of the invention. As used herein the phrase “neuromotor disorder” expressly encompasses and contemplates all the above conditions and their like without need to expressly list each condition. The plurality of sensors is configured to provide kinematic data regarding the motion and/or angle of the sensor. The sensor data is normalized and combined to generate quantifiable measurements of postural alignment and stages of partial postural control in non-ambulatory persons.

It is another object of the invention to provide a method whereby the sensor data is obtained, normalized, and used to classify movement patterns in a patient with normal development or a patient with a neuromotor disorder; the movement patterns elucidated for each being indicative of a stage of postural control achieved.

It is another object of the invention to provide a method that executes an algorithm based on kinematic data from at least one of the head or trunk or another anatomical position of humans for the purpose of quantifying postural alignment and stages of partial postural control in non-ambulatory persons.

It is another object of the invention to provide a method to detect the trunk control development process in enough detail to explore the underlying mechanisms involved during the development of partial trunk control. Embodiments of the method include the formulation of intervention plans and assessments of effectiveness of new interventions for people with normal development, or postural dysfunction, or other neuromotor diseases.

In certain embodiments a plurality of sensors is provided. The sensors are affixed at anatomical reference points. Data is collected from the sensors and correlated to anatomical segments. The position of the anatomical segments with respect to each other is then determined over lengths of time and in view of additional interventions or intervening effects. The positional data is then interpreted into a stage of anatomical control or postural control.

In certain embodiments of a method of treating a patient with a neuromotor disorder a plurality of sensors configured to capture kinematic data from a subject are provided. The sensors capture kinematic data for a proscribed length of time. The kinematic data is then converted into a postural score. A treatment plan is then altered or created based on at least one postural score. In some embodiments the kinematic data is converted into a Markov-like mode. In still other embodiments, one or more postural scores are aggregated to determine the effectiveness of a treatment or intervention. In still other embodiments, the plurality of sensors may be at least one selected from the group of: accelerometer, gyroscope, motion capture, and magnetic bearing. In other additional embodiments a first postural score is calculated without intervention and one or more subsequent postural scores are calculated following one or more interventions. In certain other embodiments the proscribed length of time is of shorter duration in a treatment setting leading to a subsequent, longer evaluative time frame. In still other embodiments, the kinematic data is converted into one or more stages of postural control. A length of time spent in each stage of postural control may then be determined as a percentage of the proscribed length of time. In still other embodiments, the kinematic data is sourced from anterior and lateral video footage.

In another embodiment of a method of treating a patient with a neuromotor disorder, a plurality of sensors are attached to a patient at proscribed anatomical reference points. Kinematic data is then captured from the plurality of sensors for a proscribed length of time. Measurements of postural alignment are then calculated. Stages of partial postural control are calculated from the measurements of postural alignment. A present stage of partial postural control may then be displayed in real-time in a treatment setting. A treatment plan is then created or altered due to at least one stage of partial postural control calculated for at least a first proscribed length of time. In some embodiments, a first intervention is provided and a stage of partial postural control is calculated for a second proscribed length of time. A treatment plan is then created or altered by comparing the stage of postural control obtained during the first proscribed length of time to the stage of postural control obtained during the second proscribed length of time. In still another embodiment, the plurality of sensors is at least one selected from the group of: accelerometer, gyroscope, motion capture, and magnetic bearing. In certain additional embodiments, one or more Markov-like models and associated postural scores are generated from one or more proscribed lengths of time performed before and after one or more interventions. In certain embodiments the first proscribed length of time is shorter than at least a second subsequent proscribed length of time. Indeed, the first proscribed length of time may be a half hour or less and the at least a second subsequent proscribed length of time is a day or more. In still other alternative embodiments the first proscribed length of time may be of short duration in a treatment setting and of longer duration in the home, school, or community environments. Thus, in an additional embodiment, a first set of kinematic data may be measured using a first plurality of sensors for a first proscribed length of time and a second set of kinematic data may be measured using a second plurality of sensors for a second, longer, proscribed length of time. The first plurality of sensors may be at least one selected from the group of: motion capture, video, and direct; and, the second plurality of sensors may be at least one selected from the group of: accelerometer, gyroscope, and magnetic bearing. In some embodiments the sensors for the first plurality of sensors may be the same as the sensors for the second plurality of sensors. In an additional embodiment, the anatomical reference points are at least two selected from the group of: head, trunk, spine, thoracic trunk, and lumbar trunk. Additional sensor locations may be used as required in view of different neuromotor diseases or sensor technologies without departing from the scope of the invention.

In an embodiment, an apparatus for the treatment of a patient with a neuromotor disorder comprises a non-transient, machine-readable storage medium encoded with a non-transitory program code for execution by a processor for generating a postural score, the program code configured for the reception of kinematic data from a subject from a plurality of sensors for a proscribed length of time; converting the kinematic data into a postural score; and, displaying the postural score. In additional embodiments the postural score may be printed or stored into memory or incorporated as part of a database. A clinician then creates or alters a treatment plan based on the postural score.

These and other objects of the invention will become apparent upon reading the detailed description below with reference to the non-limiting example embodiments provided.

### BRIEF DESCRIPTION OF THE DRAWINGS

Features and advantages of embodiments of the present invention will become apparent on reading the detailed description below with reference to the drawings, which are illustrative but non-limiting.

FIG. 1 presents examples of children with typical development and with CP demonstrating collapse stage of postural control. Histogram represents location of head with respect to midline during three (3) minutes of postural sway for a child who demonstrates only collapse.

FIG. 2 presents examples of children with typical development and with CP demonstrating rise and fall stage of postural control. Histogram represents location of head with respect to midline during three (3) minutes of postural sway for a child who demonstrates only the rise and fall stage of postural control.

FIG. 3 presents examples of children with typical development and with CP demonstrating wobble stage of postural control. Histogram represents location of head with respect to midline during three (3) minutes of postural sway for a child who demonstrates primarily wobble stage of postural control.

FIG. 4 presents examples of children with typical development and with CP demonstrating stable stage of postural control. Histogram represents location of head with respect to midline during three (3) minutes of postural sway for a child who demonstrates only the stable stage of postural control.

FIG. 5 presents an example of a child with CP demonstrating head stage of postural control.

FIG. 6 is an example of a graphic illustration of postural stages for a child with CP across twelve (12) minutes under two conditions.

FIG. 7 is an illustrative example of sensor placement for a child head sensor, child trunk sensor, infant head sensor and infant trunk sensors and showing possible anatomical placement of sensors.

FIG. 8 is an example illustration of sensor coordinates for child head sensor, child trunk sensor, infant head sensor and infant trunk sensor showing how alignment with gravity may vary across different sensors.

FIG. 9 is a graphic illustration of an example of x-y-z accelerometer head raw data, trunk raw data, head sway angle and trunk sway angle in two planes.

FIG. 10 is a process diagram demonstrating the conversion of accelerometer data to a postural score which indicates improvement or degradation of a subject's postural control under changing conditions.

FIG. 11 shows the detail of a postural classification

FIG. 12 shows the detail of a postural state function.

FIG. 13 shows the detail of a non-initial postural state function.

FIG. 14 shows the detail of an upright state function.

FIG. 15 shows the detail of rise, fall, wobble function which determines if the current postural state is “rise,” “fall,” or “wobble.”

FIG. 16 shows the detail of a WRH subroutine which determines if the current postural state is “rise,” “fall,” “wobble,” “stable,” or “head” based on the previous postural state being “wobble,” “rise,” or “head.”

FIG. 17 shows the detail of the S subroutine which determines if the current postural state is “wobble,” “head,” “stable,” or “fall” based on the previous postural state being “stable.”

FIG. 18 shows detail of the C subroutine which determines if the current postural state is “wobble,” “head,” “stable,” “collapse,” or “rise” based on the previous postural state being “collapse.”

FIG. 19 shows detail of F subroutine which determines if the current postural state is “rise,” “fall,” “wobble,” “collapse,” “head,” or “stable” based on the previous postural state being “fall.”

FIG. 20 shows detail of an AP subroutine which determines if the current state is “wobble,” “rise,” or “fall” if the trunk is tilted more prominently in the AP direction compared to the ML direction.

FIG. 21 shows detail of ML subroutine which determines if the current state is “wobble,” “rise,” or “fall” if the trunk is tilted more prominently in the ML direction compared to the AP direction.

FIG. 22 shows examples of the postural score calculation output for two different participants having two different levels of support.

### DETAILED DESCRIPTION OF THE INVENTION

All publications, patents, and patent applications mentioned in this specification are intended to be incorporated in their entirety by each and every publication, patent, or patent application that is specifically and individually referenced. To the extent that they are incorporated herein by reference in their entirety. In case of conflict between the terms herein and the terms of the incorporated references, the terms herein will prevail.

The concept for stages of upright control was first introduced by Sandra Saavedra in 2012. (Saavedra, Sandra L., Paul van Donkelaar, and Marjorie H. Woollacott. “Learning about gravity: segmental assessment of upright control as infants develop independent sitting.” Journal of Neurophysiology 108.8 (2012): 2215-2229; the entirety of which is incorporated herein by reference). Four stages of postural control termed “Collapse,” “Rise and Fall,” “Wobble,” and “Functional” were elucidated and described. The stages were initially described and created based on manual review of recorded video of postural behavior when different levels of external support were provided to typical infants during development of postural control. The stages were also demonstrated in children with moderate to severe cerebral palsy who were constrained in development of trunk control. (Saavedra, Sandra L., and Marjorie H. Woollacott. “Segmental contributions to trunk control in children with moderate-to-severe cerebral palsy.” Archives of physical medicine and rehabilitation 96.6 (2015): 1088-1097; the entirety of which is incorporated herein by reference. Prior to these two publications, other posture control researchers classified upright control based on a single segment model of trunk control. The single segment model works well for persons who have achieved upright control, but it does not allow adequate detail to document changes in trunk control for persons with partial control. Although used and described in the context of cerebral palsy patients, it is contemplated that for other neuromotor disorders that similar stages of anatomical control and/or postural control can be defined and ultimately measured via the process without departing from the spirit of the invention.

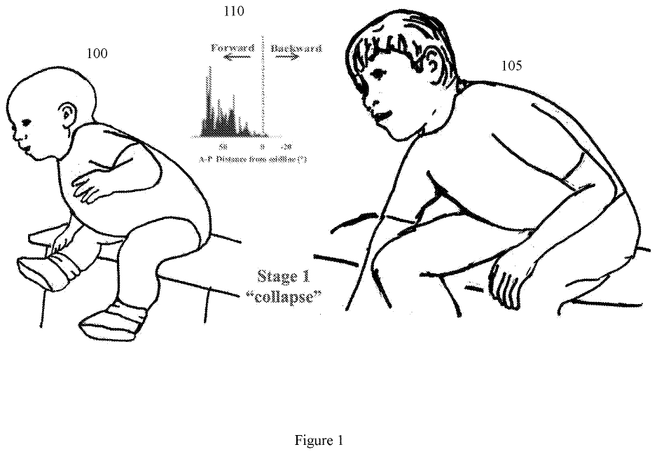

FIG. 1 presents examples of children with typical development demonstrating the collapse stage of postural control 100 and children with CP demonstrating the collapse stage of postural control 105. Collapse histogram 110 represents location of the head with respect to midline during 3 minutes of postural sway for a child who demonstrates only collapse. In children with typical development demonstrating the collapse stage of postural control 100 less than three (3) months of age, collapse is the most common postural stage when upright positioning is attempted. This is also common in children with the most severe disabilities caused by a variety of neurologic diagnoses. This stage of postural control represents an inability of the child to oppose the pull of gravity, thus they end up collapsing forward to the end of their range of motion. Electromyography (EMG) activity has been illustrated at this stage however the amount and timing of the activity is not adequate to return the child to upright alignment. Collapse histogram 110 shows that when positioned upright the child falls away from midline and spends increased time near the edge of range of motion and is unable to return to vertical alignment on their own.

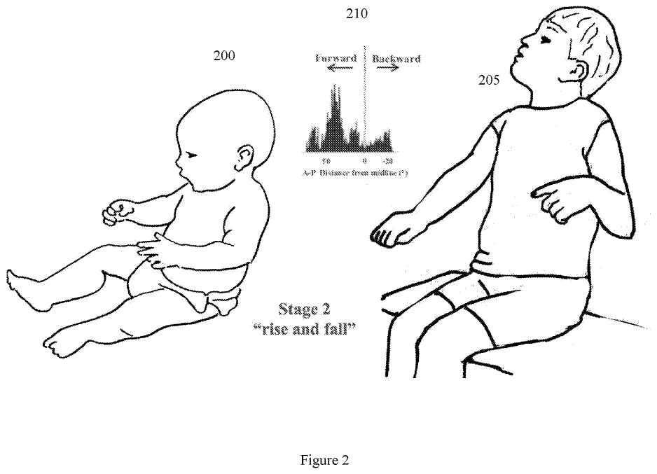

FIG. 2 presents examples of children with typical development demonstrating the rise and fall stage of postural control 200 and children with CP demonstrating rise and fall stage of postural control 205. Rise/fall histogram 210 represents the location of the head with respect to midline during three (3) minutes of postural sway for a child who demonstrates only the rise and fall stage of postural control. Children with typical development demonstrating the rise and fall stage of postural control 200 at 4-5 months of age most often demonstrate rise and fall postural behavior when upright positioning is attempted, this is also seen in children with neurologic disabilities who have some partial ability to control their trunk muscles. This stage of postural control represents an inability of the child to stabilize in vertical alignment but the child is able to rise towards midline from either an anterior or posterior leaning position. The rise/fall histogram 210 shows an example of data from a child who primarily used rise and fall behaviors when attempting to remain upright. When upright position was attained the child was unable to sustain the position and fell forward or backward, spending increased time near the edges of range of motion between attempts to come upright. For purposes of the algorithm used in an example embodiment of the invention presented below, rise and fall are not paired together. The algorithm evaluates each type of behavior independently. Fall behavior is not dependent on rise, nor is rise dependent on fall. Thus, a fall does not necessarily need to be followed by a rise and vice versa, a rise does not need to be followed by a fall. The algorithm allows for more detailed information on each of these behaviors and allows them to transition to any other behavioral type.

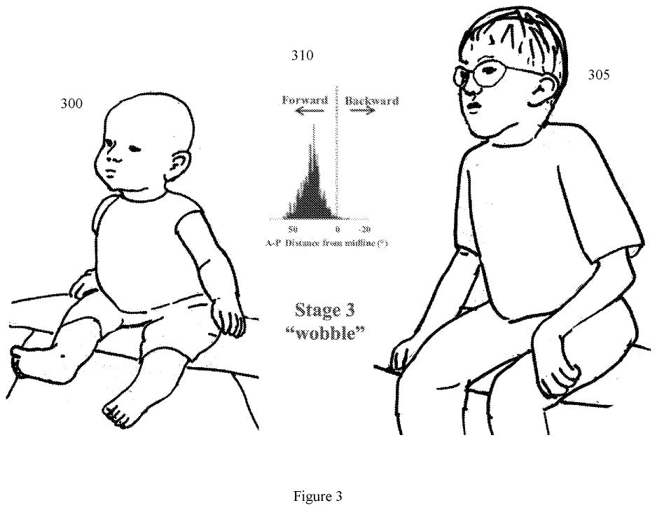

FIG. 3 presents examples of children with typical development demonstrating wobble stage of postural control 300 and children with CP demonstrating wobble stage of postural control 305. Children with typical development demonstrating wobble stage of postural control 300 at 6-7 months of age most often demonstrate wobble postural behavior when upright positioning is attempted, this is also seen in children with neurologic disabilities who can sit partially upright. This stage of postural control represents emerging postural control. The child can stabilize in an upright but not completely vertical alignment. Postural sway has greater range than typical such that the child tends to lean a bit forward and slowly wobbles side to side and forward and back but the child remains upright. Wobble histogram 310 represents location of the head with respect to midline during three (3) minutes of postural sway for a child who demonstrated primarily the wobble stage of postural control. The normal distribution of the histogram suggests that at this stage there is a central set point that the child keeps returning to; however, that set point is not vertically aligned.

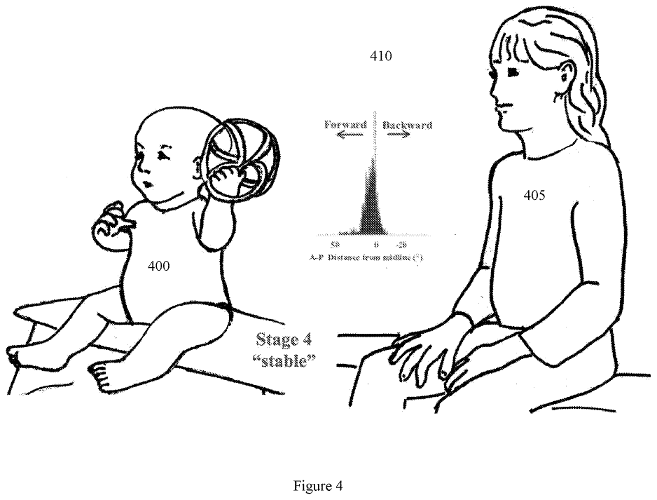

FIG. 4 is a schematic showing examples of children with typical development demonstrating stable stage of postural control 400 and children with CP demonstrating stable stage of postural control 405. Children with typical development demonstrating stable stage of postural control 400 demonstrate stable postural behavior at approximately 7-8 months of age when independent sitting is achieved. This is also seen in some children with neurologic disabilities who can sit independently. This stage of postural control represents control of upright sitting position. This is the goal that is achieved with full control. Stable histogram 410 represents location of the head with respect to midline during three (3) minutes of postural sway for a child who demonstrated primarily the stable stage of postural control. The normal distribution of the histogram suggests that at this stage there is a central set point that the child keeps returning to that is centered around vertical upright. The range of sway is narrower than in the previous wobble stage.

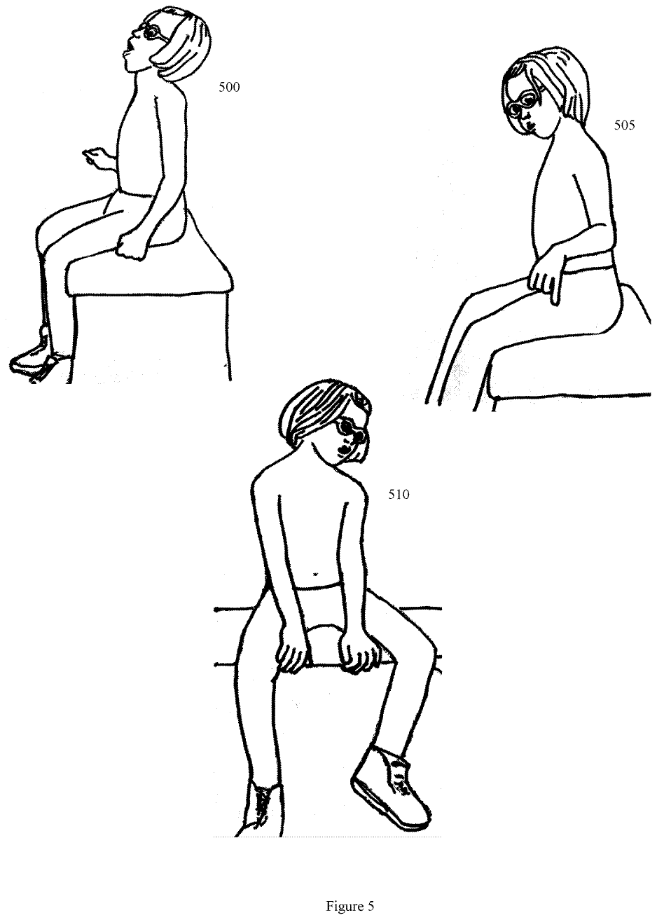

FIG. 5 is a schematic showing examples of a child with CP demonstrating head stage of postural control. In this stage the trunk is upright however the child is not able to keep the head stable and vertically aligned. The bead may exhibit backward fall 500, forward fall 505, or sideways fall 510. This is sometimes observed in children who have cortical visual impairment that results in holding the head at angled positions to adjust the visual field. Other children demonstrate this stage when given adequate support for the trunk to be upright but who lack the postural control necessary to hold their head stable and vertical.

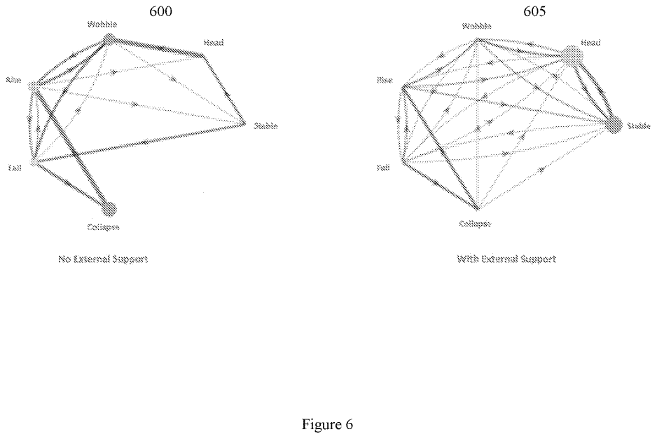

FIG. 6 is an example of a graphic illustration of postural stages for a child with CP across 12 minutes under two conditions. Markov model with no support 600 shows the results with the child sitting on a bench with pelvic support straps and no external support. Markov model with support 605 shows the results with the addition of an external trunk support placed according to the child's level of trunk control. The density and direction of the arrows represent the frequency and direction of postural transitions. The size of the node represents time spent in each state. These images allow a visual representation of the full repertoire of postural behavior and how it changes under different conditions. More time spent to the right and upper side of the image shows improved postural control while more time and transitions towards the left and lower portions of the image indicate poor control.

In certain embodiments a plurality of sensors is provided. The sensors are affixed at anatomical reference points. Data is collected from the sensors and correlated to anatomical segments. The position of the anatomical segments with respect to each other is then determined over proscribed lengths of time and in view of additional interventions or intervening effects. The positional data is then interpreted into a stage of anatomical control. The plurality of sensors is configured to provide kinematic data regarding the motion and/or angle of the sensor. The sensor data is normalized and combined to generate quantifiable measurements of postural alignment and stages of partial postural control in the form of a postural score for non-ambulatory persons.

In certain embodiments the sensor encompasses one or more discrete devices capable of measuring the position of the sensor with respect to the field of gravity. In certain other embodiments the sensors may be part of a motion capture system and may, for example, comprise reflective dots used in an infrared based motion capture system. In still other embodiments, one or more distinctive markings may be placed on the subject either directly or on the clothing and measured in a captured video frame. Sensor types may include accelerometers, gyroscopes, magnetic field direction (e.g., orientation with respect the magnetic field of Earth), and strain gauges. It is readily appreciable by one of ordinary skill in the art that the amount of data points generated from such systems is typically well beyond what could be ordinarily done with pen-and-paper, particularly in a real-time or near-real time evaluation. Sensors may be worn on the body, sewn into clothing, placed into special pockets, etc. without departing from the spirit of the invention. Such sensors may be called “wearable sensors” or “WS” though they may not indeed necessarily be worn directly (e.g., as in the case of direct motion capture). Wearable sensors, just as the name implies, are integrated into wearable objects or directly with the body in order to help monitor health and/or provide clinically relevant data for care.

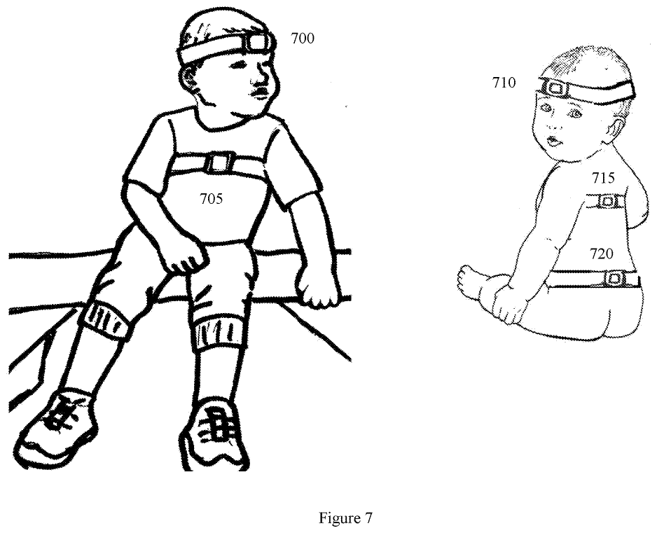

FIG. 7 is an illustrative example of sensor placement for a child head sensor 700, child trunk sensor 705, infant head sensor 710, infant thoracic trunk sensor 715, and infant lumbar trunk sensor 720 showing possible anatomical placement of sensors. Data from a minimum of two sensors; child head sensor 700 or infant head sensor 710 and at least one child trunk sensor 705 or infant thoracic trunk sensor 715 or infant lumbar trunk sensor 720, is required to determine stage of postural control. Data can be obtained from additional trunk sensors. The trunk sensors can be placed on anterior surface of the body, as shown with child trunk sensor 705, or posterior as shown with infant thoracic trunk sensor 715 and infant lumbar trunk sensor 720. Child head sensor 700 and infant head sensor 710 should ideally be placed anteriorly, centered on the forehead or placed on each side of the head for estimated center of mass of the head.

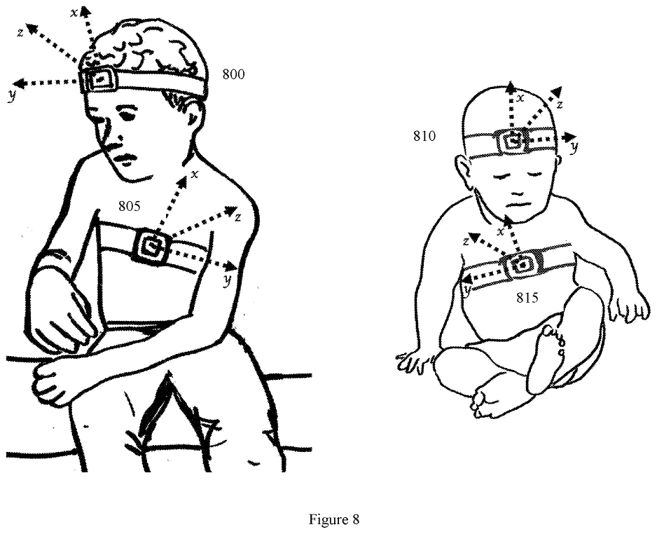

FIG. 8 is an illustration showing examples of sensor coordinates for child head sensor coordinate axes 800, child trunk sensor coordinate axes 805, infant head sensor coordinate axes 810, and infant trunk sensor coordinate axes 815 demonstrating how alignment with gravity and coordinate axes may vary across different sensors. If the lower body leans or twists away from upright the upper trunk and head must produce compensatory alignment in order to stay upright and balanced. This method allows tracking of orientation and sway characteristics of compensatory processes across multiple segments of the spine. Knowledge of how the various segments are managed allows stages of postural control to be determined and better understanding of whether the child is participating in motor learning for new control or in compensatory strategies that will prevent motor learning.

In an embodiment of the invention, eight children, between the ages 23 months and 13 years old with moderate to severe neuromotor dysfunction and deficit in sitting postural control were recruited. Four APDM Opal sensors (APDM Inc., Portland, OR, USA) were placed on the anterior head, anterior trunk and dorsum of the left and right wrists to monitor movement patterns of their respective segments to which they were attached. The dimensions of these devices are 43.7×39.7×13.7 mm (L×W×H) and have mass less than 25 g. The devices have a tri-axial component of accelerometer, gyroscope and magnetometer and can measure acceleration, angular velocity and position in space at a rate of up to 128 Hz. In some embodiments the sensor may be a strain gauge. In still other embodiments, data from a strain gauge is combined with data from an accelerometer to provide a measurement of trunk segment orientation.

To serve as a control and validation of the process, two Canon FS400 camcorders on tripods were placed anterior and laterally to capture a full body image of each child. Humans were trained to observe movement patterns on a video and classify the child's behavioral responses according to the above listed stages of control. The behavior coders judged which behavioral stage the child was using from observations of both anterior and lateral camera views. Simultaneous kinematic data from wearable sensors strapped to the child's head and torso provided the kinematic data that were fed into the algorithm for similar classification of stages.

A segmental assessment of trunk control (“SATCo”) was performed to determine specific level of trunk support the child needed. A child was strapped and seated onto a SATCo bench and evaluated until they lost control of their posture and were unable to maintain upright. After this level was determined, the child was given a rest break prior to the first session. This information was used to provide each participant their appropriate level of trunk support during the first session of play.

Over the course of at least two sessions, the participants were strapped and seated on a SATCo bench with pelvic strapping for stability and provided an external trunk support at the lowest level of the trunk where control was demonstrated during SATCo (i.e. upper thoracic, mid thoracic, or lower thoracic). In the first session, an adaptive support, R82 Meerkat stander (R82, Inc., Matthews, NC, USA) was used to provide adequate trunk support and depending on the height of the child, 1 or 2 adjustable bands were placed around the child's trunk to support and stabilize their spine. Once the child was given trunk support, the WS were placed on the child and video recording began. When all the sensors were in place, the child's left arm was raised and lowered five times by the researcher to signal the beginning of the session.

Four different activities were used to encourage unilateral and bilateral arm movements for a total of 12 minutes. These activities included playing with a suspended rubber ball, placing and removing pegs from a peg board, reaching for and throwing plastic balls and reaching for bubbles. The beginning and end of each trial was marked by raising the child's arm five times. After finishing a session of play, a rest break was given to the child while the researcher removed the sensors and uploaded the data to Mobility Lab, Motion Studio (APDM Inc., Portland, OR, USA). For the second session, the child was asked to perform the same tasks without an adaptive support. For safety, a therapist sat behind the participant and provided manual support at the pelvis and guarded the child from injury in the event of rapid movements. When needed, brief support was given to the upper trunk to help the child return to an upright position.

### Data Reduction and Analysis

Raw data were uploaded from the sensors to a laptop immediately after each session. First, the data were converted to h5 files and saved to Mobility Lab, Motion Studio software. The h5 files contained information across three planes, x, y, z from the accelerometer, gyroscope and magnetometer.

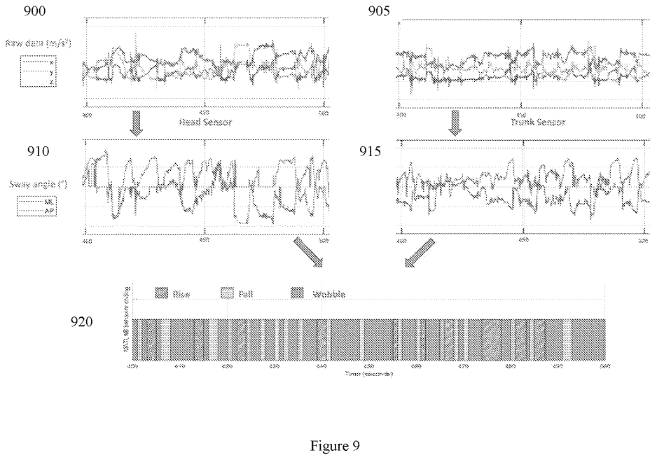

FIG. 9 is a graphic illustration showing head raw data 900 and trunk raw data 905, that is transformed into head sway angle 910 and trunk sway angle 915 in two planes (AP and ML) and final translation through the algorithm to determine the postural stage time series A20. The sensor x-y-z data may come from a variety of sensor types (such as accelerometry, optical or magnetic kinematics, magnetometry, video segmental analysis). Sway angle is determined through angle calculation 1010 and plotted across time for head sway angle 910 and trunk sway angle 915 resulting in output of the child's unique postural stage time series 920. The postural stages displayed by the child may be altered through a variety of conditions including the child's state of arousal, environmental stimuli with respect to visual or auditory input or provision of external postural support that is provided manually or through support devices.

To assess changes in overall movement, the data from the tri-axial components of the accelerometer were analyzed using custom MATLAB (Mathworks, Inc., Natick, MA, USA) algorithms. The raw data was then filtered through a type 2 low pass Chebyshev filter (5 Hz cutoff frequency) to eliminate any background noise. The head and trunk angles were then derived from the filtered data. Once the data was filtered, additional algorithms were created to create an output csv file to assign numerical codes and colors to various behaviors as obtained from video behavior coding. This was then used to overlap video and wearable sensor data to time match each data set. In certain embodiments, raw sensor data may be processed, evaluated, and stored in a non-transitory computer-readable medium onboard a platform bearing the sensor. In still other embodiments, multiple sensors may be attached and/or otherwise operably connected to a control module configured to receive and process the data.

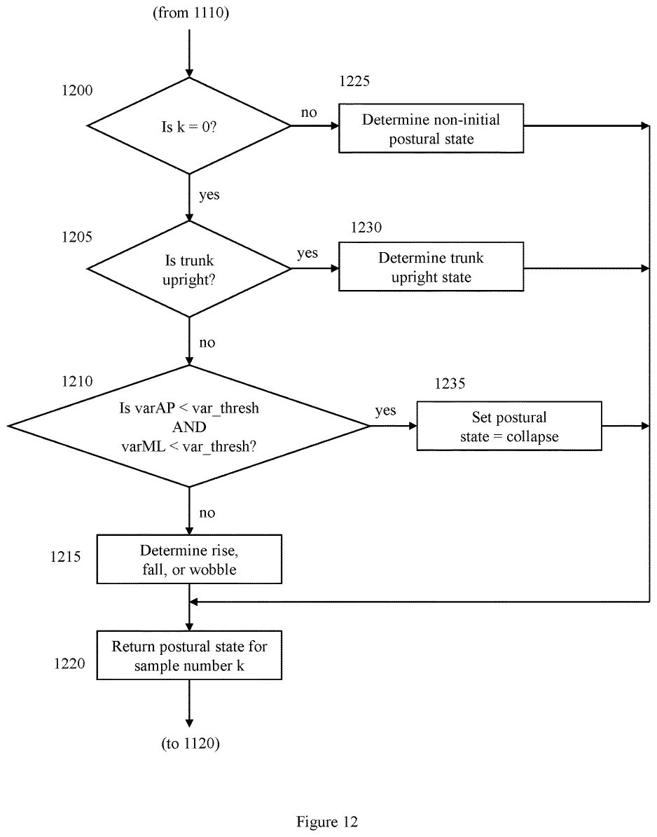

The data retrieved from the WS was cut based on the start and end times of each session. The start time was identified through a MATLAB code. In certain embodiments, time-synchronized data were fed from a non-transient computer readable medium to a controller configured to:
At an initial time point using an initial batch of orientation data determine an initial state (see FIG. 12). Possible outputs states were “Wobble”-W; “Stable”-S; “Collapse”-C; “Rise”-R; “Fall”-F; and, a determination that the trunk was upright while the head was not, “Trunk upright, head not upright”-H. It is not possible for each state to transition to each other state. In some instances, passing from one state requires passage through one or more other states before arriving at a new state. For example, a stable state S may go through a fall F state before arriving at a collapse C state. A subject may then move to a rise R state and then back to a stable S state.
At a second time point a second set of orientation data are combined with the initial state to generate a second state. (See FIG. 12.) This process is iteratively repeated until either all data are processed (i.e., time has run out) or the process is manually halted. The final output is a time series of postural states (as shown in the bottom graph of FIG. 9).

After the generation of the time series of postural states, the data may then be analyzed to determine percentage of time spent in each state, the effects of various interventions, and the overall duration each child spends in each state throughout the day. The data may be plotted as a color-coded graphic with each behavior represented by a different color. In other embodiments, Markov-like models such as those in FIG. 6 are generated indicating duration in a particular state and transition from one state to another. A first Markov-like model for a patient may be compared with at least a second or more Markov-like model (either later in time or after a treatment intervention, or both) to determine the efficacy of an intervention or the overall progress of a child. For example, a first Markov-like model may show a child with a relatively greater period of time spent in wobble, rise or collapsed states (indicating a lack of motor control) whereas a later Markov-like model may indicate a relatively increased time spent in the head and/or stable conditions (indicating a greater degree of motor control). The treatment plan of a child may then be altered or held the same.

Comparison of the outcomes between human monitored encoding and the present invention indicated that video behavior coding using human interpretation evaluating each frame of captured video was cruder. This is partly due to the less refined sampling rate of the video at 60 Hz versus 128 Hz for the wearable sensor. There were some instances where human video behavior coders were inaccurate presumably due to pauses in concentration, distraction, or fatigue. The amount of time involved was more than 100-fold greater for human-interpreted video behavior coding. Further, human interpretation was found to be subjective. In contrast, according to an embodiment of the invention, algorithmic processing to classify 12 minutes of data took less than 30 seconds compared to 45 minutes or longer for the human interpreter to classify the same data. Moreover, the algorithm produced more accurate and repeatable classifications than the human video behavior coder. Subsequent comparisons of human encoders evaluating the same footage showed little agreement with each other. In contrast, the present invention is capable of objectively returning the same results for the same session evaluated.

In certain embodiments, a postural score may be calculated and used as the basis of comparison. Postural scores may be calculated before and after treatments or interventions and used to evaluate the success or failure of said treatments or interventions. Practitioners could then alter the plan of treatment for a patient based on the postural score. In still other embodiments, postural scores may be aggregated across patients and used to evaluate the effectiveness of proposed treatments or interventions. It is an object of the invention that postural scores generated are done so in an objective, repeatable, fashion of which human evaluators are not capable, thus improving overall reliability in evaluating treatment effects while leading to increased successful patient outcomes.

Embodiments of the invention, such as those herein described may utilize the below-described algorithmic processes to classify subjects into the previously described stages of postural control. In certain embodiments of the invention a postural score is generated that may be used to compare a patient before and after treatment or intervention. In other embodiments, output scores of patients may be compared to each other to determine the effectiveness of one or more treatments or interventions on a patient population. Intervention, in addition to its usual meaning, may be viewed as an action taken to improve a situation, especially a medical disorder. Interventions that may benefit from the invention include improved information and awareness during treatment sessions, quantification of the effectiveness of different positioning devices and increased awareness of the frequency and quality of upright opportunities across a day or across different environments. Treatment sessions could be conducted by a plurality of health and educational professionals including, but not limited to, physical, occupational, or speech therapists, adaptive equipment specialists, teachers, coaches, etc. Embodiments of the invention utilized during treatment sessions may include real time or summary feedback. Real time feedback will allow the clinician to adjust manual or external support or alter the task and immediately see the results in terms of the quality of upright control. Summary feedback could provide quantification across treatment sessions to examine variability from one session to another and hopefully improved opportunities for upright control as treatment sessions progress.

In still other embodiments, on a larger time scale the disclosed procedure could provide full or partial day data that would allow analysis of the number of opportunities the child has for practicing upright control throughout the day at home, in the community, or at school. Similarly, the procedure may be carried out in as little as one minute or several seconds worth of evaluation. In still other embodiments, sensors may be worn for weeks, months, or years with data periodically collected and evaluated and postural scores calculated.

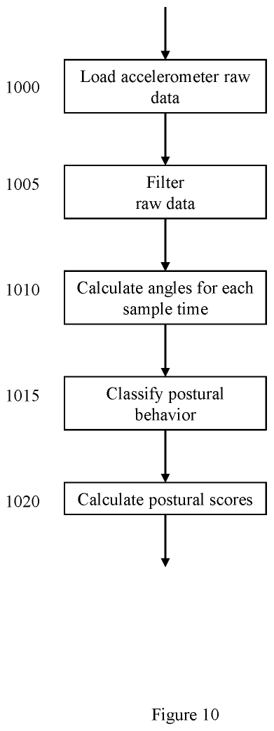

FIG. 10 is a process diagram demonstrating the conversion of accelerometer data to a postural score which indicates improvement or degradation the postural control of a subject patient under changing conditions. Importation step 1000 demonstrates the importation of raw accelerometer data from the x, y, and z axes measured in m/s² over the entire session (˜12 minutes) into a computer program running the algorithm. The raw data was collected using a sampling frequency of 128 Hz in this embodiment, while other sampling frequencies are possible. Filter 1005 is a filtering algorithm to remove high frequency noise to ensure that linear accelerations were less prominent in the data than gravitational effects. The filter was a 12th order type 2 lowpass Chebyshev filter with a 2 Hz bandwidth and 40 dB of stopband attenuation. Other types of filters and parameters are possible to achieve the same effect. This filtering step effectively eliminated linear acceleration and noise, leaving only the gravitation component of the acceleration along each axis. Angle calculation 1010 performs the calculation of accelerometers' (and thus the wearer's) orientation angles in the anterior-posterior (AP) plane (front/back) and mediolateral (ML) plane (left/right) at each sampling instant. The angles in each plane, θ_{AP} and θ_{ML}, were calculated using the following formulas:

$${θ}_{ML} = \{ \begin{matrix}a tan 2 ( y , x ) , & if ( x > 0 ) ∧ ( \vert z \vert > \vert y \vert + {acc}_{thresh} ) \\ 0 , & if ( \vert x \vert < {acc}_{min} ) ∧ ( \vert y \vert < {acc}_{min} )\end{matrix} \qquad ( 1 )$$

$${θ}_{AP} = \{ \begin{matrix}a tan 2 ( z , x ) , & if ( x > 0 ) ∧ ( \vert y \vert > \vert z \vert - {acc}_{thresh} ) \\ 0 , & if ( \vert x \vert < {acc}_{min} ) ∧ ( \vert z \vert < {acc}_{min} )\end{matrix} \qquad ( 2 )$$

The values used were accₜₕᵣₑₛₕ=0.1×9.81 m/s² and accₘᵢₙ=0.4× 9.81 m/s². The calculations in equations (1) and (2) were performed using data from the child trunk sensor 705 and child head sensor 700 resulting in θ_{T}_{_{AP}}, θ_{T}_{_{ML}}, θ_{H}_{_{AP}}, and θ_{H}_{_{ML}} respectively. Postural classification 1015 is the classification of postural behavior into one of the postural states (stable, wobble, collapse, head, rise, fall) based on θT_{AP}, θ_{T}_{_{ML}}, θ_{H}_{_{AP}}, and θ_{H}_{_{ML}}. This step produces the postural stage time series 920 which can then be aggregated into models similar to Markov model with no support 600 or Markov model with support 605. From this postural stage time series 920 two forms of information can be extracted: the amount of time spent in each state and how the test subject transitions between states in a given session.

The time spent in each state is visually depicted by the size of the nodes in Markov model with no support 600 and Markov model with support 605, which is also numerically represented for one example session as shown in Table 1. The Time (seconds) column is the total time spent in each postural state during the session. The Proportion column is the value in the Time (seconds) column divided by the total session time (952 s).

TABLE 1

| Postural State | Time (seconds) | Proportion |
|---|---|---|
| Stable | 4 | 0.004202 |
| Head | 30 | 0.031513 |
| Wobble | 258 | 0.271008 |
| Rise | 238 | 0.25 |
| Fall | 195 | 0.204832 |
| Collapse | 227 | 0.238445 |
| Total | 952 | 1 |

How many times the test subject transitions between states is visually depicted as the thickness of the connecting curves in Markov model with no support 600 and Markov model with support 605, which is also represented by a transition matrix shown in Table 2 for one example session.

TABLE 2

| | | Next State | | | | | |
|---|---|---|---|---|---|---|---|
| | | Stable | Head | Wobble | Rise | Fall | Collapse |
| Current | Stable | 0 | 1 | 2 | 0 | 1 | 0 |
| State | Head | 0 | 0 | 11 | 1 | 3 | 0 |
| | Wobble | 3 | 7 | 0 | 44 | 82 | 0 |
| | Rise | 1 | 7 | 90 | 0 | 63 | 0 |
| | Fall | 0 | 0 | 33 | 37 | 0 | 78 |
| | Collapse | 0 | 0 | 0 | 79 | 0 | 0 |

The transition matrix in Table 2 indicates that during the session, the test subject transitioned from wobble to stable three times, and from rise to stable once. The diagonal is always zero since only transitions to a different state are considered. Normalizing the above transition matrix is achieved by dividing each value by the total number of transitions (543, which is the sum of all entries in the matrix). See Table 3. Summing across each row results in 1 since a state must transition to some other state.

TABLE 3

| | | Next State | | | | | |
|---|---|---|---|---|---|---|---|
| | | Stable | Head | Wobble | Rise | Fall | Collapse |
| Current | Stable | 0 | 0.25 | 0.5 | 0 | 0.25 | 0 |
| State | Head | 0 | 0 | 0.73333 | 0.066667 | 0.2 | 0 |
| | Wobble | 0.022059 | 0.051471 | 0 | 0.32353 | 0.60294 | 0 |
| | Rise | 0.006211 | 0.043478 | 0.55901 | 0 | 0.3913 | 0 |
| | Fall | 0 | 0 | 0.22297 | 0.25 | 0 | 0.52703 |
| | Collapse | 0 | 0 | 0 | 1 | 0 | 0 |

2D scoring calculation 1020 is the process of converting the time series of behaviors into a pair of scores, labeled as “state score” and “transition score” quantifying the subject's postural control over the entire session (˜12 minutes) into two values between zero and one inclusive (with higher values indicating better postural control).

The state score was calculated as follows

$$SS = \frac{{w}_{stable} {T}_{stable} + {w}_{head} {T}_{head} + {w}_{wobble} {T}_{wobble}}{max ( {w}_{stable} , {w}_{head} , {w}_{wobble} )} \qquad ( 3 )$$

where W_{stable}, W_{head}, and w_{wobble} are weights indicating the relative importance of the respective stable, head, and wobble states. T_{stable}, T_{head}, and T_{wobble} are the proportional times spent in the stable, head, and wobble states respectively using the data from Table 1 for example. The state score calculated in equation (3) is normalized so that only the relative and not absolute magnitudes of the weights affect the score. That is, using weights values (2, 0.5, 1) and (4, 1, 2) will result in the same score. SS is a measure of how much time was spent in a “good” state (stable, head, wobble) with the weights indicating the relative goodness of the good states.

The transition score TS was calculated as follows

$$TS = \frac{{w}_{stable} {S}_{stable} + {w}_{head} {S}_{head} + {w}_{wobble} {S}_{wobble}}{max ( {w}_{stable} , {w}_{head} , {w}_{wobble} )} \qquad ( 4 )$$

$$where$$

$${S}_{stable} = \frac{{P}_{rise , stable} + {P}_{fall , stable} + {P}_{collapse , stable}}{{S}_{all}} \qquad ( 5 )$$

$${S}_{head} = \frac{{P}_{rise , head} + {P}_{fall , head} + {P}_{collapse , head}}{{S}_{all}} \qquad ( 6 )$$

$${S}_{wobble} = \frac{{P}_{rise , wobble} + {P}_{fall , wobble} + {P}_{collapse , wobble}}{{S}_{all}} \qquad ( 7 )$$

$${S}_{all} = {\sum }_{\begin{matrix}i ∈ \{ rise , fall , collapse \} \\ j ∈ \{ rise , fall , collapse , stable , head , wobble \}\end{matrix}} {P}_{i , j} \qquad ( 8 )$$

P_{i,j} in equation (8) is the probability of transition from state i to state j and the weights W_{stable}, W_{head}, and w_{wobble} are the same as described above. The probability values are taken from Table 3 for example. As with SS, TS calculated in equation (4) is normalized so that it only depends on the relative values of the weights. TS is measure of how often “bad” states (rise, fall, collapse) transition to “good” states (stable, head, wobble).

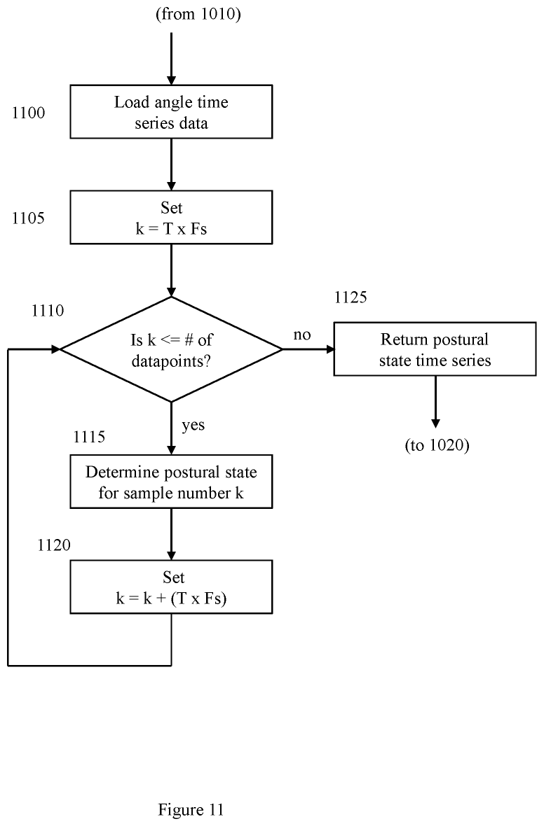

Continuing on, FIG. 11 shows the detail of postural classification 1015. Angle loading 1100 is the process of loading the AP and ML angles for the trunk and head that were produced by angle calculation 1010. Counter initialization 1105 is the process of initializing a counter variable “k” to T×Fs, where T is the observation time interval (1 second) and Fs is the sample rate of the sensor (128 Hz). Counter check 1110 checks to see if the current counter value k is less than the number of datapoints in the session. If the condition is true, then the current timestep is within the time for the session (˜12 minutes) and postural state function 1115 executes. If the condition is false, then the current timestep is beyond the end time of the dataset and return postural time series 1125 executes. Postural state function 1115 is the process of assigning a behavior (stable, wobble, collapse, head, rise, fall) to the current timestep. Counter increment 1120 is the step that increments the counter “k” by the number of datapoints in the observation time interval. Return postural time series 1125 is the process of returning the postural stage time series 920 to the 2D scoring calculation 1020.

FIG. 12 shows the detail of postural state function 1115. Initial counter check 1200 is conditional check to see if the counter k is T×Fs (which indicates the first datapoint is being processed). If the condition is true, trunk upright check 1205 executes. If the condition is false, non-initial postural state function 1225 executes. Trunk upright check 1205 is a conditional check to see if the trunk is upright. “Trunk upright” is defined mathematically as θ_{T}<θ_{T}_{ₘₐₓ}, where

$${θ}_{T} = {tan}^{- 1} ( \sqrt{{( tan {θ}_{{T}_{ML}} )}^{2} + {( tan {θ}_{{T}_{AP}} )}^{2}} ) \qquad ( 9 )$$

and θ_{T}_{ₘₐₓ}=20°. Trunk upright can be visually depicted as the trunk being within a 20° vertical cone. If the trunk upright condition is true, then upright state function 1230 is executed. If the trunk upright condition is false, then variance check 1210 executes. Variance check 1210 is a conditional check to determine if the trunk is moving by examining the variance of θ_{T}_{_{AP}} and θ_{T}_{_{ML}} over a time interval of 1 second. If both variances are less than a set threshold) (10°, then collapse state 1235 executes, which assigns the current postural state as “collapse.” If either variance is more than a set threshold) (10°), then rise, fall, wobble function 1215 executes. Rise, fall, wobble function 1215 is the process to determine if the postural state at the current sample time is rise, fall, or wobble. Finally, kth postural state return 1220 returns the current determined value of the postural state to the calling function and the process proceeds to counter increment 1120.

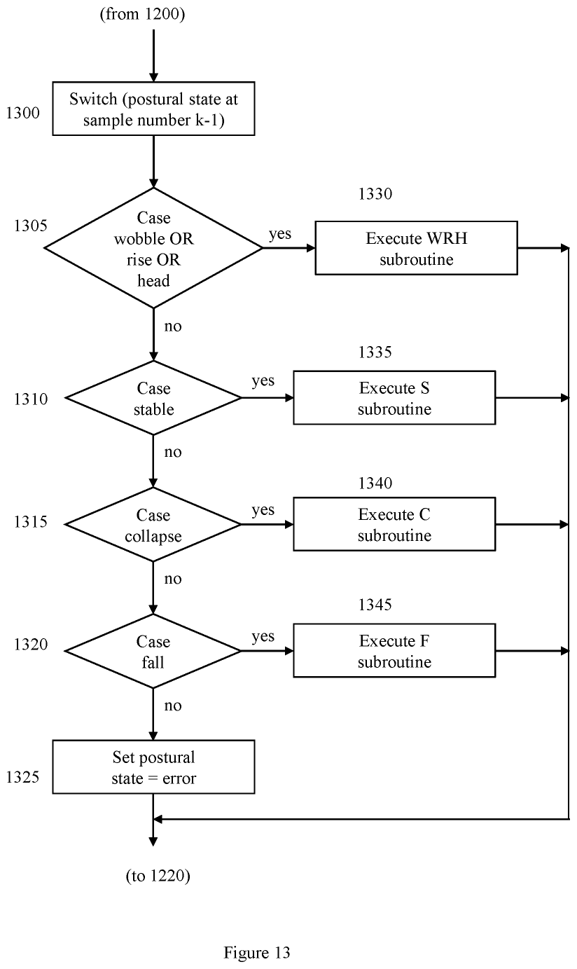

FIG. 13 shows the detail of non-initial postural state function 1225. Switch statement 1300 checks the assigned postural state for the previous sample time (at k-1), which may be any of six states (stable, wobble, collapse, head, rise, fall). Case wobble rise head 1305 is a conditional check to see if the previous postural state was either wobble, rise, or head. If this condition is true, then wobble-rise-head (“WRH”) subroutine 1330 executes. If this condition is false, then case stable 1310 is executed to check to see if the previous postural state was stable. If this condition is true, then S subroutine 1335 executes. If this condition is false, then case collapse 1315 is executed to check to see if the previous postural state was collapse. If this condition is true, then C subroutine 1340 executes. If this condition is false, then case fall 1320 is executed to check to see if the previous postural state was fall. If this condition is true, then F subroutine 1345 executes. If the condition is false, then error state 1325 executes which assigns the current postural state as “error.”

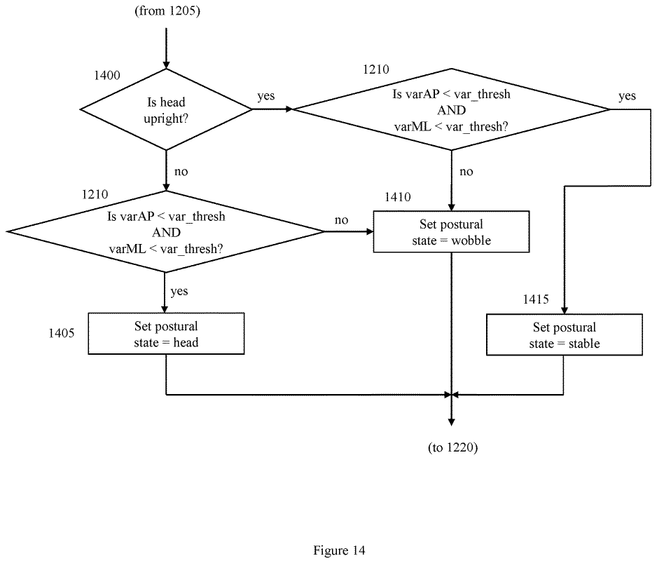

FIG. 14 shows the detail of upright state function 1230. Head upright check 1400 is a conditional to check if the head is upright. “Head upright” is defined mathematically as θ_{H}<θ_{H}_{ₘₐₓ}, where

$${θ}_{H} = {cos}^{- 1} ( \frac{{TH}^{T}}{\sqrt{1 + {( tan {θ}_{{T}_{ML}} )}^{2} + {( tan {θ}_{{T}_{AP}} )}^{2}} \sqrt{1 + {( tan {θ}_{{H}_{ML}} )}^{2} + {( tan {θ}_{{H}_{AP}} )}^{2}}} ) \qquad ( 10 )$$

$$T = [ \begin{matrix}1 & - tan {θ}_{{T}_{ML}} & - tan {θ}_{{T}_{AP}}\end{matrix} ] \qquad ( 11 )$$

$${H}^{T} = \{ \begin{matrix}{[ \begin{matrix}1 & - tan {θ}_{{H}_{ML}} & - tan {θ}_{{H}_{AP}}\end{matrix} ]}^{T} , & if ( {θ}_{{H}_{ML}} \leq 90 ° ) ∧ ( {θ}_{{H}_{AP}} \leq 90 ° ) \\ {[ \begin{matrix}- 1 & tan {θ}_{{H}_{ML}} & - tan {θ}_{{H}_{AP}}\end{matrix} ]}^{T} , & if ( {θ}_{{H}_{ML}} > 90 ° ) ∧ ( {θ}_{{H}_{AP}} \leq 90 ° ) \\ {[ \begin{matrix}- 1 & - tan {θ}_{{H}_{ML}} & tan {θ}_{{H}_{AP}}\end{matrix} ]}^{T} , & if ( {θ}_{{H}_{ML}} \leq 90 ° ) ∧ ( {θ}_{{H}_{AP}} > 90 ° )\end{matrix} \qquad ( 12 )$$

and θ_{H}_{ₘₐₓ}=30°. Head upright can be visually depicted as the head being within a 30° cone protruding out of the top of the trunk. Whether the condition is true or false, variance check 1210 is executed. If the outcome of variance check 1210 is false, then wobble state 1410 is executed and the current postural state is set to “wobble.” If the outcome of variance check 1210 is true, then either head state 1405 is executed (if the head is not upright) or stable state 1415 is executed (if the head is upright).

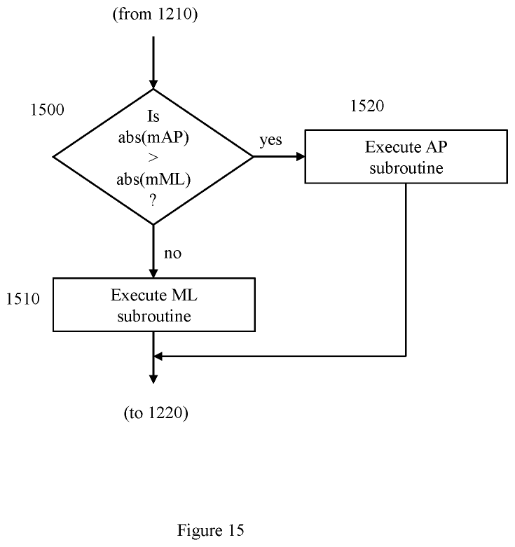

FIG. 15 shows the detail of rise, fall, wobble function 1215 which determines if the current postural state is “rise,” “fall,” or “wobble.” First direction check 1500 checks if the magnitude of the mean of θ_{T}_{_{AP}} over the previous 1 second time interval is greater than the magnitude of the mean of θ_{T}_{_{ML}} over the same 1 second time interval. If the condition is true, it indicates that the trunk tilt in the AP direction is more prominent than in the ML direction and AP subroutine 1520 executes. If the condition is false, it indicates that the trunk tilt in the ML direction is more prominent than in the AP direction and ML subroutine 1510 executes.

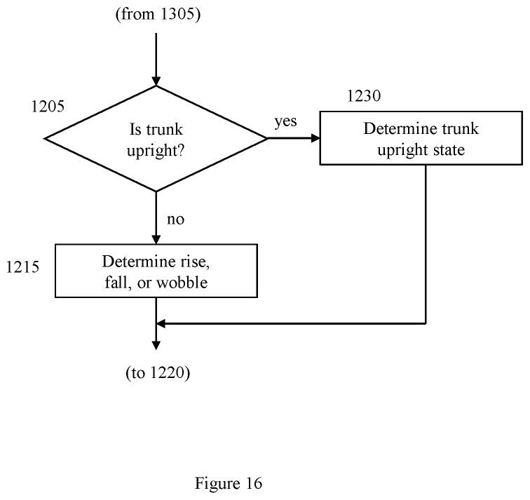

FIG. 16 shows the detail of WRH subroutine 1330 which determines if the current postural state is “rise,” “fall,” “wobble,” “stable,” or “head” based on the previous postural state being “wobble,” “rise,” or “head.” Trunk upright check 1205 is a conditional check to see if the trunk is upright. If the condition is true, then upright state function 1230 executes. If the condition is false, then rise, fall, wobble function 1215 executes.

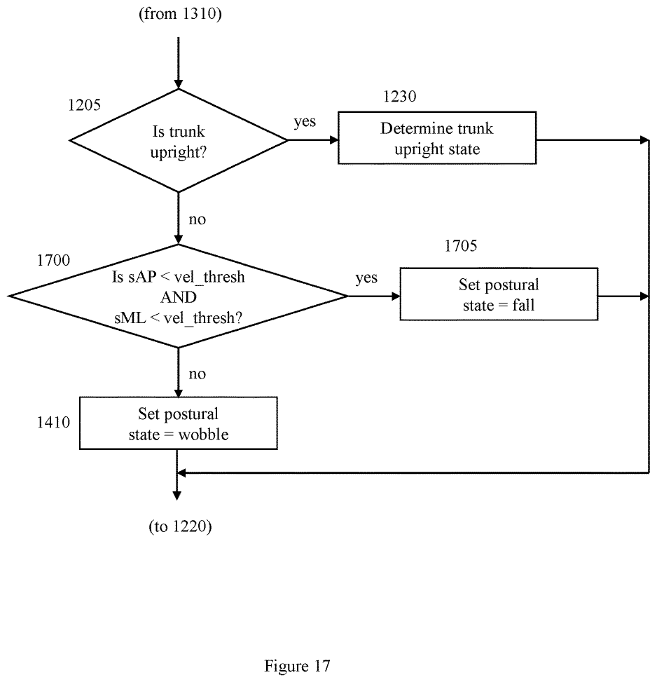

FIG. 17 shows the detail of S subroutine 1335 which determines if the current postural state is “wobble,” “head,” “stable,” or “fall” based on the previous postural state being “stable.” Trunk upright check 1205 is a conditional check to see if the trunk is upright. If the condition is true, then then upright state function 1230 executes to determine if the current postural state is “wobble,” “head,” or “stable.” If the condition is false, then velocity check 1700 determines if the slope of the best fit line to the previous 1 second of both θ_{T}_{_{AP}} and θ_{T}_{_{ML}} are both below a threshold (5°/s). If both values are below the threshold, then fall state 1705 executes, which sets the current postural state to “fall.” If either value is above the threshold, then wobble state 1410 is executed and the current postural state is set to “wobble.”

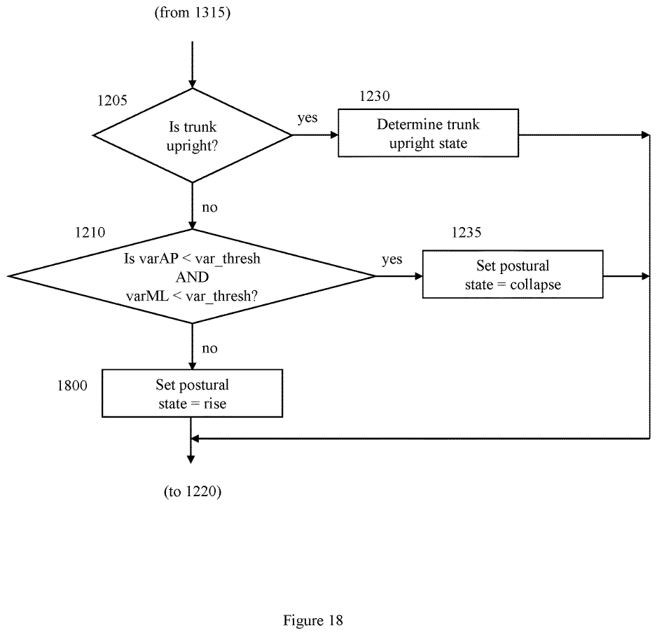

FIG. 18 shows detail of C subroutine 1340 which determines if the current postural state is “wobble,” “head,” “stable,” “collapse,” or “rise” based on the previous postural state being “collapse.” Trunk upright check 1205 is a conditional check to see if the trunk is upright. If the condition is true, then then upright state function 1230 executes to determine if the current postural state is “wobble,” “head,” or “stable.” If the condition is false, then variance check 1210 executes to determine if the trunk is moving or stationary. If the condition is true, meaning the trunk is stationary, then collapse state 1235 executes, which assigns the current postural state as “collapse.” If the condition is false, meaning the trunk is moving, then rise state 1800 executes, which assigns the current postural state as “rise.”

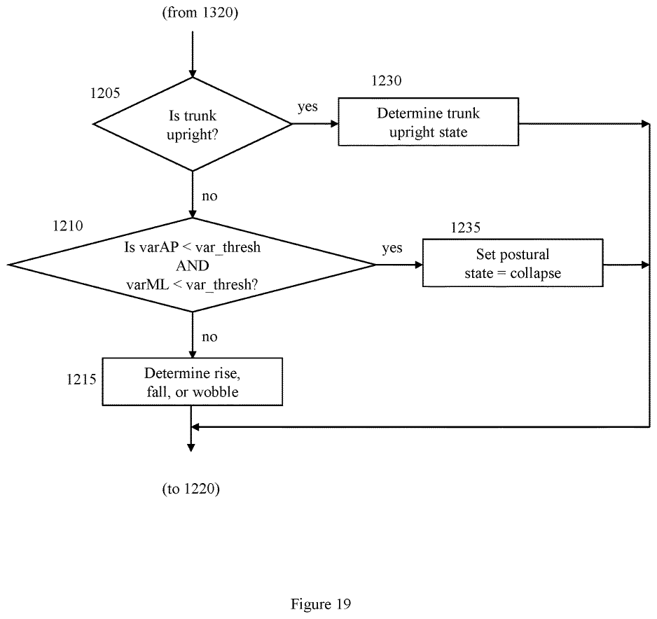

FIG. 19 shows detail of F subroutine 1345 which determines if the current postural state is “rise,” “fall,” “wobble,” “collapse,” “head,” or “stable” based on the previous postural state being “fall.” Trunk upright check 1205 is a conditional check to see if the trunk is upright. If the condition is true, then then upright state function 1230 executes to determine if the current postural state is “wobble,” “head,” or “stable.” If the condition is false, then variance check 1210 executes to determine if the trunk is moving or stationary. If the condition is true, meaning the trunk is stationary, then collapse state 1235 executes, which assigns the current postural state as “collapse.” If the condition is false, meaning the trunk is moving, then rise, fall, wobble function 1215 executes to determine if the current state is “rise,” “fall,” or “wobble.”

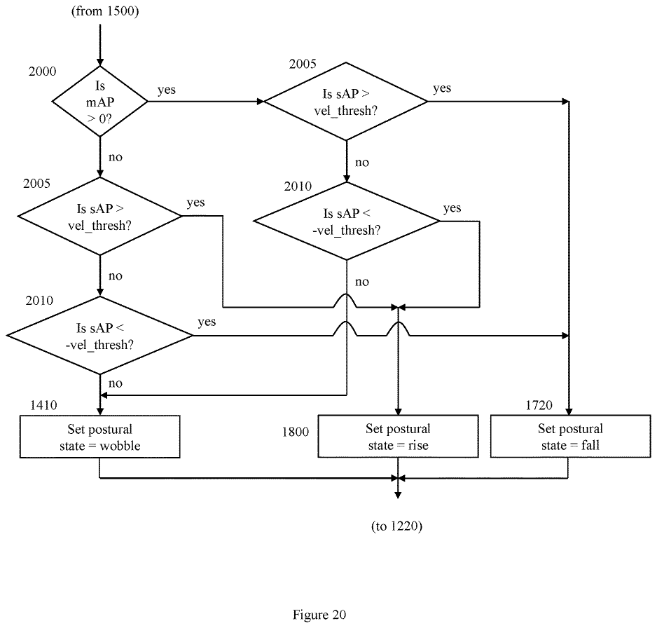

FIG. 20 shows detail of AP subroutine 1520 which determines if the current state is “wobble,” “rise,” or “fall” if the trunk is tilted more prominently in the AP direction compared to the ML direction. First AP mean check 2000 is a conditional check to determine if the mean of θ_{T}_{_{AP}} over the previous 1 second was positive or negative, which indicates if the trunk was leaning to the front or back. Whether the condition is true or false, AP velocity check 2005 is a conditional check to determine if the slope of the best fit line to the previous 1 second of both θ_{T}_{_{AP}} is above a threshold (5°/s) indicating trunk motion in the AP direction. If the condition is true, then either fall state 1705 executes (if AP mean check 2000 returned true), or rise state 1800 executes (if AP mean check 2000 returned false). If the condition is false, the AP negative velocity check 2010 is a condition check to determine if the slope of the best fit line to the previous 1 second of both θ_{T}_{_{AP}} is below a threshold (−5°/s) indicating trunk motion in the AP direction. If the condition is true, then either rise state 1800 executes and the current postural state is set to “rise” (if AP mean check 2000 returned true), or fall state 1705 executes and the current postural state is set to “fall” (if AP mean check 2000 returned false). If the condition is false, then wobble state 1410 executes and the current postural state is set to “wobble.”

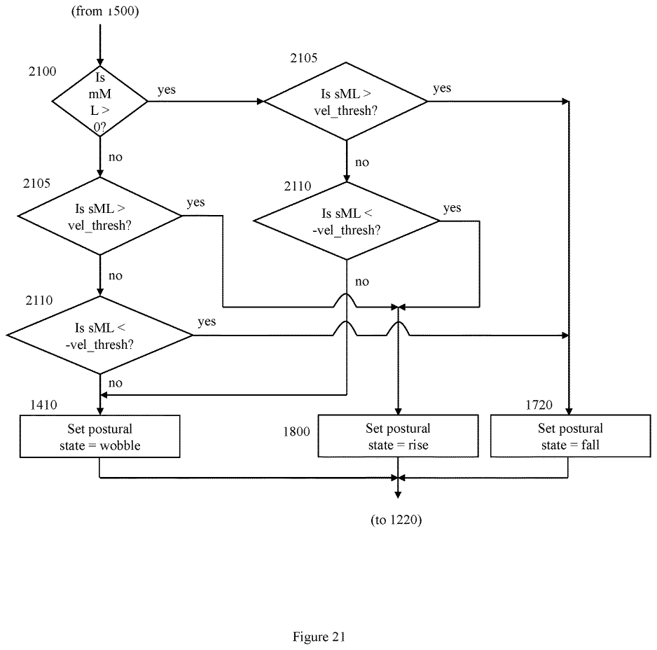

FIG. 21 shows detail of ML subroutine 1510 which determines if the current state is “wobble,” “rise,” or “fall” if the trunk is tilted more prominently in the ML direction compared to the AP direction. First ML mean check 2100 is a conditional check to determine if the mean of HTML over the previous 1 second was positive or negative, which indicates if the trunk was leaning to the left or right. Whether the condition is true or false, ML velocity check 2105 is a conditional check to determine if the slope of the best fit line to the previous 1 second of both θ_{T}_{_{ML}} is above a threshold (5°/s) indicating trunk motion in the ML direction. If the condition is true, then either fall state 1705 executes (if ML mean check 2100 returned true), or rise state 1800 executes (if ML mean check 2100 returned false). If the condition is false, the ML negative velocity check 2110 is a condition check to determine if the slope of the best fit line to the previous 1 second of both θ_{T}_{_{AP}} is below a threshold (−5°/s) indicating trunk motion in the ML direction. If the condition is true, then either rise state 1800 executes and the current postural state is set to “rise” (if ML mean check 2100 returned true), or fall state 1705 executes and the current postural state is set to “fall” (if ML mean check 2100 returned false). If the condition is false, then wobble state 1410 executes and the current postural state is set to “wobble.”

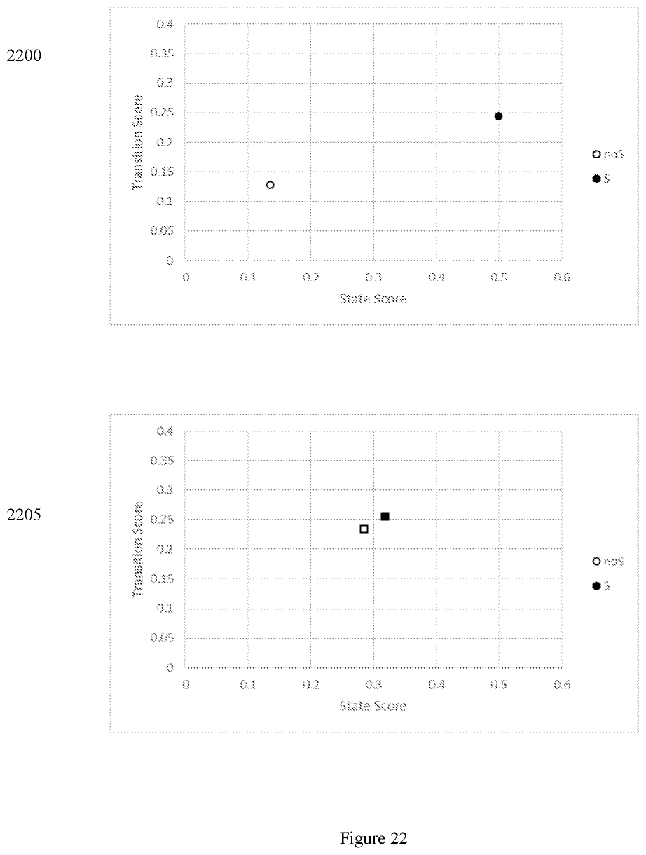

FIG. 22 shows examples of the 2D scoring calculation 1020 output for two different participants having two different levels of support. In an example of a postural score comparison, 2D score example A 2200, demonstrates a participant who showed significant improvement in postural control compared to 2D score example B 2205. In both cases the hollow marker indicates that no external support was provided to the participant, while the filled marker indicates that external support was provided. The presence or absence of external support is an example of different conditions. Improvement is manifested as a transition from lower left to upper right in the plots. Significant improvement is manifested as greater distance between the markers. 2D score example A 2200 shows the state score improving from 0.12 to 0.5 and transition score improving from 0.13 to 0.24. 2D score example B 2205 shows the state score improving from 0.28 to 0.32 and the transition score improving from 0.23 to 0.26.

In some embodiments, such as for usage with different neuromotor diseases, different definitions particular to the disorder may be used for some behaviors without departing from the spirit of the invention. This allows for further evaluation of potential behaviors and customization of the method for special cases such as children with hypotonia who respond with slower movements but lean into support surfaces.

It is appreciated that certain embodiments of the invention may be carried out on a general-purpose computing device. In some instances, such a computing device may comprise: a processor, a memory device for storing program code or other data, a display device, and one or more input devices. In certain circumstances the processor may be a microprocessor or microcontroller-based platform capable of executing computer code from a non-transitory computer-readable medium. Such a non-transitory medium can include random access memory (RAM), read only memory (ROM), flash memory, optical memory, or other storage such is known in the art. It should be appreciated that although a processor and memory devices are possible implementations, further embodiments can also be implemented using one or more application-specific integrated circuits (ASIC's) or other hard-wired devices, or using mechanical devices (collectively, and/or individually referred herein as a “processor”). Furthermore, although the processor and memory device in certain embodiments reside in a discrete computer, it is possible to provide some or all of their functions from an off-site device such as a network server configured for communication such as over a local area network (LAN), wide area network (WAN), Internet connection, microwave link, and the like. The processor and memory device are generally referred to as a “computer” or “controller.” In certain embodiments one or more of the above-described steps may be carried out on board the sensors attached to the patient or partially processed before final processing using one or more processors.

For purposes of the present invention, the term “real time” refers to the conventional meaning of the term “real time,” i.e., reporting, depicting, or reacting to events at the same rate and sometimes at the same time as they unfold, rather than storage or processing at a later time. In some embodiments postural scores are calculated for set times before and after the application of an intervention during a treatment session. The intervention may be maintained or altered as a result of the score. Subsequently, a longer-term evaluation (days, weeks, months, years, etc.) may occur to more thoroughly vette the effectiveness of the chosen intervention.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. It is further understood that the sequence of certain steps may be re-arranged without departing from the broader scope of the invention. Further, certain sub-routines and their use are conditionally dependent on predecessor conditions and may or may not be used in any given execution of the process.

Since certain changes may be made in the above-described invention, without departing from the spirit and scope of the invention herein involved, it is intended that all of the subject matter of the above description shown in the accompanying drawings shall be interpreted merely as examples illustrating the inventive concept herein and shall not be construed as limiting the invention.

What is claimed is:

1. A method of treating a patient with a neuromotor disorder comprising:
providing a plurality of sensors configured to capture kinematic data from a subject;
capturing kinematic data for a proscribed length of time;
converting the kinematic data into a postural score; and,
altering or creating a treatment plan based on at least one postural score.

2. The method of claim 1 further comprising:
converting the kinematic data into a Markov-like model.

3. The method of claim 1 wherein one or more postural scores are aggregated to determine the effectiveness of a treatment.

4. The method of claim 1 wherein the plurality of sensors is at least one selected from the group of: accelerometer, gyroscope, motion capture, and magnetic bearing.

5. The method of claim 1 wherein a first postural score is calculated without intervention and one or more subsequent postural scores are calculated following one or more interventions.

6. The method of claim 1 wherein the proscribed length of time is of shorter duration in a treatment setting leading to a subsequent, longer, evaluative time frame.

7. The method of claim 1 wherein the kinematic data is converted into one or more stages of postural control.

8. The method of claim 7 further comprising:
determining the length of time spent in each stage of postural control as a percentage of the proscribed length of time.

9. The method of claim 1 wherein the kinematic data is sourced from anterior and lateral video footage.

10. A method of treating a patient with a neuromotor disorder comprising:
attaching a plurality of sensors to a patient at proscribed anatomical reference points;
capturing kinematic data from the plurality of sensors for a proscribed length of time;
calculating measurements of postural alignment;
calculating from the measurements of postural alignment stages of partial postural control;
displaying in real-time in a treatment setting the present stage of partial postural control; and,
creating or altering a treatment plan due to at least one stage of partial postural control calculated for at least a first proscribed length of time.

11. The method of treating a patient with a neuromotor disorder of claim 10 further comprising:
providing a first intervention;
calculating a stage of partial postural control for a second proscribed length of time;
creating or altering a treatment plan as a result of comparing the stage of postural control obtained during the first proscribed length of time to the stage of postural control obtained during the second proscribed length of time.

12. The method of treating a patient with a neuromotor disorder of claim 10 wherein the plurality of sensors is at least one selected from the group of: accelerometer, gyroscope, motion capture, and magnetic bearing.

13. The method of treating a patient with a neuromotor disorder of claim 10 wherein one or more Markov-like models and associated postural scores are generated from one or more proscribed lengths of time performed before and after one or more interventions.

14. The method of treating a patient with a neuromotor disorder of claim 10 wherein the first proscribed length of time is shorter than at least a second subsequent proscribed length of time.

15. The method of treating a patient with a neuromotor disorder of claim 14 wherein the first proscribed length of time is a half hour or less and at least a second subsequent proscribed length of time is a day or more.

16. The method of treating a patient with a neuromotor disorder of claim 10 further comprising:
measuring a first set of kinematic data using a first plurality of sensors for a first proscribed length of time;
measuring a second set of kinematic data for a second plurality of sensors for a second, longer, proscribed length of time.

17. The method of treating a patient with a neuromotor disorder of claim 16, wherein the first plurality of sensors are at least one selected from the group of: motion capture, video, and direct; and, wherein the second plurality of sensors is at least one selected from the group of: accelerometer, gyroscope, and magnetic bearing.

18. The method of treating a patient with a neuromotor disorder of claim 10 wherein the anatomical reference points are at least two selected from the group of: head, trunk, spine, thoracic trunk, and lumbar trunk.

19. An apparatus for the treatment of a patient with a neuromotor disorder comprising:
a non-transient, machine-readable storage medium encoded with a non-transitory program code for execution by a processor for generating a postural score, the program code configured for:
receiving from a plurality of sensors for a proscribed length of time kinematic data from a subject;
converting the kinematic data into a postural score; and,
displaying the postural score.

20. The apparatus for the treatment of a patient with a neuromotor disorder of claim 19, wherein a clinician creates or alters a treatment plan based on the postural score.
